(19) United States
(12) Patent Application Publication
(10) Pub. No.: US 2002/0198495 A1
(43) Pub. Date: Dec. 26, 2002

(54) Automatic pole syringe

(75) Inventors: Thomas Hogan, Marietta, GA (US); Kimberly Quinn, Kenansville, NC (US)

Correspondence Address: William B. Dyer III, Esq., 263 Skyland Drive, Roswell, GA, 30075, US

(73) Assignee: VAC-PAC, Inc.

(21) Appl. No.: 09/888,200

(22) Filed: Jun. 23, 2001

(51) Int. Cl. A61M 1/00

(52) U.S. Cl. 604/151; 606/116

(57) ABSTRACT

An automatically loading pole syringe has a grip affixed to a first end and an automatically loading syringe needle assembly affixed to a second end. The pole syringe carries a bottle mount for mounting a medicine bottle. A medicine delivery tube interconnects the medicine bottle to the automatically loading syringe needle assembly. An embodiment of the present invention includes an ink carriage for carrying an ink source, the ink carriage being oriented to allow the ink source captured therein to discharge ink in a direction proximal to a needle carried by the syringe needle assembly.

### TECHNICAL FIELD

The present invention relates to syringes for providing medicine injections to animals.

### BACKGROUND

Recent outbreaks of animal diseases such as “Foot and Mouth” and BSE (also known as “Mad Cow” disease) are threatening both the livelihoods of food animal producers and the safety of one of the critical staples of world food supply. Now—more than ever—reliable and efficient mechanisms for administering vaccines and other medicines to food animals are necessary.

In recent years, numerous mechanisms have been developed that have improved the way medicines are delivered to food animals. For instance, the commercially available VAC-MARC syringe—elements of which are taught in issued U.S. Pat. No. 5,961,494 and allowed U.S. patent application Ser. No. 09/389,774 (both to the inventor Hogan herein and specifically incorporated herein by reference), available through VAC-PAC Corporation of Kenansville, N.C. (1-800-4VACPAC). This family of syringes allow the simultaneous marking of a food animal at the same time an injection is applied to the animal. As described in these referenced patents, this technique allows verification that an animal has received an injection, as well as indicating where on the animal the injection was applied, an important fact in effectively delivering medicines to animals.

Another important development in animal medicine delivery is disclosed in pending U.S. patent application Ser. No. 09/477,262, also to Hogan. This system, available commercially as the VAC-TRAC Verification System (“VTVS”) provides—among other things—a mechanism to automatically and electronically record the unique identity of an animal that has received an injection. As disclosed in that pending application, each food animal is given an electronic identification device (“EID”) that emits a unique identification signal. When the VAC-TRAC syringe is actuated and delivers the injection to the animal, a reader on the syringe reads the unique identification signal relating to the animal and transmits to a computer database the identification of the animal and the medicine applied to the animal, thus creating a verifiable database of medical administrations to food animals.

Notwithstanding these important developments, most animal injections—including injections from each of these previously discussed systems—are delivered by a hand-actuated syringe. That is, the syringe is held in one hand and actuated when opposing syringe handles are squeezed together. Obviously, such arrangement requires the individual applying the injections to be in very close proximity to the animal receiving the injection.

It is not always desirable for the individual applying the injection to be in close proximity to the animal. In the case of a relatively short animal, such as a hog, such proximity requires the individual to bend over for long periods of time, potentially causing physical injury or fatigue. In this or other similar situations, such proximity may not be desirable because of the rambunctious nature of the animals in question. Additionally, such proximity may not be desirable because of sanitary reasons. In any event, it would be desirable for a mechanism to allow an individual providing injections to do so from some distance away from the animals.

This need has been recognized and addressed by the commercial availability of a syringe known as the “long shot”, available through Koehn Marketing, P.O. Box 577, Watertown, S. Dak., 57201-0677, 800-658-3998. This “pole syringe” generally comprises a typical syringe mechanism affixed to the end of a pole. Thus, an individual can apply an injection from a distance ranging from two to four feet (or longer, depending on the pole length) from the animal. Though this syringe addresses some of the concerns listed above, it only allows administration of a single medicine dose to a single animal before “reloading” is required. This solution is highly inefficient, given the large number of food animals in a typical herd that would need injection.

Another shortcoming of this product is that it does not mark the animal as it is injected, and thereby does not automatically indicate that the animal received the injection.

### BRIEF SUMMARY OF THE INVENTION

These and other shortcomings relating to the use of syringes, generally, and pole syringes, specifically, are satisfied by the present invention. More specifically, the present invention discloses an automatically loading pole syringe comprising a syringe pole having a length between a first end and a second end. A grip is affixed to the first end of the syringe pole and an automatically loading syringe needle assembly affixed to the second end.

A bottle mount for mounting a medicine bottle to the syringe pole is integrally incorporated therein. The present invention also includes a medicine delivery tube having a first tube end adapted for connection to the medicine bottle, and a second tube end connected to the syringe needle assembly. An embodiment of the present invention also includes an ink carriage for carrying an ink source, the ink carriage being oriented to allow the ink source captured therein to discharge ink in a direction proximal to a needle carried by the syringe needle assembly.

### BRIEF DESCRIPTION OF THE DRAWINGS

FIG. 1 depicts an exemplary embodiment of the present invention.

### DETAILED DESCRIPTION OF EXEMPLARY EMBODIMENTS

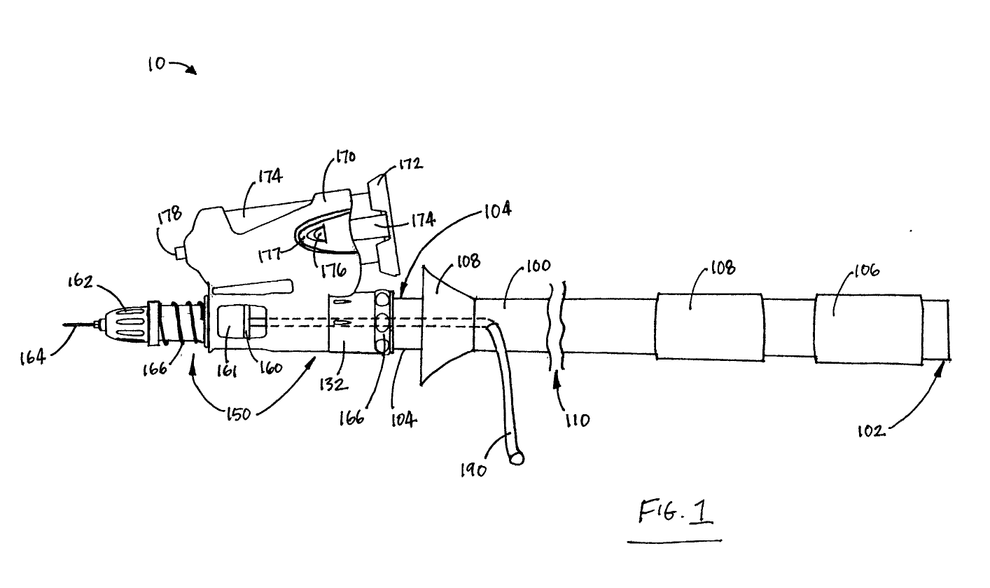

FIG. 1 depicts an exemplary embodiment of the automatically loading pole syringe (“pole syringe”) 10. The pole syringe 10 comprises, generally, a syringe pole 100, an automatically loading syringe needle assembly (“syringe”) 150, and an ink carriage 170.

More specifically, the syringe pole 100 is manufactured of a lightweight material such as polyvinylchloride (PVC) tubing, lightweight aluminum or the like. In the depicted embodiment, a cylindrical shape is illustrated. The syringe pole 100 has a first end 102 and a second end 104. Generally, the length of the syringe pole 100 is determined by the distance between the first end 102 and the second end 104, although in an alternate embodiment, the syringe pole 100 is adjustable in length by a wide variety of different mechanisms well known to those skilled in the art. A break 110 is illustrated in FIG. 1 to depict the likelihood that the syringe pole 100 may be longer—in terms of scale—than actually depicted in FIG. 1.

The first end 102 of the syringe pole 100 carries a grip 106. The grip 106 may be a knurled portion of the syringe pole 100, or it may be a flexible grip member made of a material such as rubber, etc. One purpose of the grip is to allow the user of the pole syringe 10 to maintain a firm grasp on the syringe pole 100. In the depicted embodiment, the grip 106 is simply wrapped about the syringe pole 100. Other physical configurations of the grip 106 are also specifically contemplated, such as a configuration in which the grip 106 includes an integral grip shield to protect the gripping hand of the user of the pole syringe 10.

Another optional feature depicted in FIG. 1 is the second grip 108. Having largely the same function as the grip 106, the second grip 108 allows the user to securely grip the pole syringe 10 with two hands, thereby making it easier for the user to apply an injection to a specific location and causing less user fatigue.

The second end 104 of the syringe pole 100 is functionally connected to the syringe needle assembly (syringe) 150 in a removable “screw-type” fashion to facilitate cleaning and repair. The syringe 150 comprises a syringe plunger 160, a syringe dosage chamber 161 (also known as an interchangeable volume component), a syringe needle fastener 162, and a needle 164. The dosage chamber 161 is threaded into a syringe collar 132 for easy removal for cleaning or exchange for a dosage chamber 161 of a different size.

The depicted embodiment includes a syringe head (not shown) that is hollow, for delivery of medicine to the syringe, and further includes a syringe nipple. The syringe nipple is integral to the syringe head and is sized to securely receive a hose such as a medicine delivery tube. Medicine is delivered to the hollow interior cavity of the syringe head via the medicine delivery tube that is connected to a medicine source and may be partially (or entirely) captured within the syringe pole 100.

The syringe pole 100 interacts with the syringe, and is further connected to the plunger 160. The dosage adjust valve 166 threadably engages the plunger 160. In order to vary the amount of the dosage, the dosage adjust valve 166 is rotated, thereby changing the position of the plunger 160 within the dosage chamber 161 and thereby modifying the dosage of medicine that can be received into the syringe dosage chamber 161.

The syringe plunger 160 slides within the dosage chamber 161. An O-ring (not shown) disposed about the syringe plunger 160 creates a liquid-tight seal between the periphery of the syringe plunger 160 and the interior wall of the dosage chamber 161. The syringe plunger 160 has a check valve (not shown) within its interior axial conduit that allows liquid to pass only in the direction toward the needle end of the syringe 150.

The syringe dosage chamber 161 is ideally formed of a translucent or transparent material and is secured at its first end to the syringe collar 132. The dosage chamber 161 may be scored with incremental graduations to assist a user in dosage measurements. At its second end, the dosage chamber 161 removably receives a syringe needle fastener 162. The syringe needle fastener 162 is fitted to capture a needle 164. A check valve (not shown) is fitted within the syringe needle fastener 162 to allow liquid flow only out of the needle 164.

When actuated, the plunger 160 moves forward within the dosage chamber 161, the check valve within the plunger 160 closes to force vaccine in the dosage chamber 161 through the check valve within the needle fastener 162 and out through the needle 164. When the plunger 160 moves rearward within the dosage chamber 161, the check valve within the needle fastener 162 closes to preclude fluid or air being drawn into the dosage chamber 161 through the needle 164. Simultaneously, the check valve within the plunger 160 opens so that vaccine is drawn into the dosage chamber 161 through the nipple and the syringe head.

After adjustment of the dosage adjust valve 166 to regulate the amount of medicine loaded into the syringe for an injection, the pole syringe 10 is thrust axially toward the animal to be injected. The needle 164 penetrates the carcass of the animal. When the needle 164 is fully inserted, the syringe needle fastener 162 comes into contact with the carcass. Because of the relative size and shape of the syringe needle fastener 162, it does not penetrate the carcass. As thrust is continually exerted upon the pole syringe 10 in the direction of the animal, the resistance of the animal carcass to the syringe needle fastener forces compression of the syringe spring 166.

As the syringe spring 166 compresses, the syringe pole 100 and the syringe collar 132 move in the direction of the animal. Eventually, the syringe collar 132 compresses the syringe spring 166 completely and comes to rest. Meanwhile, the syringe pole 100 continues to advance.

Continuing advancement of the syringe pole 100 advances both the plunger 160 and the marking trigger 108 that is attached to the syringe pole 100. As the plunger 160 advances, the medicine previously drawn into the syringe dosage chamber 161 is injected into the animal. As the marking trigger advances, it makes contact with the can détente 172 and the ink dispenser 174 is moved forward within the ink carriage 170. This forward action of the ink dispenser 174 triggers discharge of ink or another appropriate marking substance from the ink dispenser 174 via a discharge orifice 178 in the direction of the location of the injection.

The ink dispenser 174 may take any number of forms well known to those skilled in the art of marking substance containers, including, but not limited to, a canister, ajar, a tube, or the like. Further, the specific form of ink dispenser 174 may be dependent upon the type of ink being utilized. For instance, a pressurized canister maybe used to store ink that is suspended in, or in the form of, a compressed gas. Alternatively, a structure such as that used to store household caulk may be used to store liquid ink suitable for the particular application.

The ink carriage 170 may be rotatably interconnected to the syringe collar 132 of the syringe 150. The critical aspects of this rotatable interconnection are the ability of the ink carriage 170 to be rotated about the syringe collar 132, and the ability of the rotatable interconnection therebetween to hold the ink carriage 170 in whatever position it has been rotated to.

The can détente 172 is described with great specificity in pending U.S. patent application Ser. No. 09/848,495, also to the inventors of the present invention, and is specifically incorporated by reference herein. The can détente 172 includes at least one latch hook 176. When the latch hook 176 is inserted into the ink carriage 170, it is caught by the latch catch 177 within the ink carriage 170. This action secures the can détente 172 (and, importantly, the ink dispenser 174 attached thereto) within the ink carriage 170.

Importantly, the pole syringe 10 is automatically loading. By the term “automatically loading” it is specifically contemplated that the syringe 150 is connected to a medicine source (not shown). As previously discussed, medicine such as a vaccine is delivered to the syringe 150 by a medicine delivery tube 190 attached to a syringe nipple, the syringe nipple being attached to the syringe head. The opposite end of the medicine delivery tube 190 is attached to a medicine source.

The medicine source will most often be a resilient container such as those commonly used to transport and store medicines. In a preferred embodiment, the medicine source will be attached to the pole syringe by a bottle mount in a secure but removable fashion, so that it will remain secure during use, but can be easily removed when empty or when the pole syringe 10 is ready for cleaning. In another preferred embodiment, the medicine source may be attached to the user of the pole syringe—most likely to an arm (via an armband), to the belt (via a clip) or in a backpack-type carrier. In such a configuration, the medicine delivery tube 190 is long enough to make the connection between the medicine source and the syringe 150 and to allow the user freedom of movement.

The foregoing description of an embodiment of the present invention and certain variations thereof is not intended to limit the scope of the present invention. Rather, the scope of the present invention is dictated by the claims appended hereto.

I claim:

1. An automatically loading pole syringe, comprising:
a. a syringe pole having a length between a first end and a second end;
b. a grip affixed to the first end;
c. an automatically loading syringe needle assembly affixed to the second end of the syringe pole;
d. a bottle mount for mounting a medicine bottle to the syringe pole;
e. a medicine delivery tube having a first tube end and a second tube end, the first tube end adapted for connection to the medicine bottle and the second tube end connected to the syringe needle assembly; and
f. an ink carriage for carrying an ink source, the ink carriage oriented to allow the ink source to discharge ink in a direction proximal to a needle carried by the syringe needle assembly.

2. The automatically loading pole syringe of claim 1, wherein the length of the syringe pole is adjustable.

3. The automatically loading pole syringe of claim 1, wherein the syringe needle assembly contains an interchangeable volume component.

4. An automatically loading pole syringe, comprising
a. a syringe pole having a length between a first end and a second end;
b. a grip proximal to the first end;
c. an automatically loading syringe needle assembly removably attached to the second end of the syringe pole;
d. a bottle mount for mounting a medicine bottle to the syringe pole; and
e. a medicine delivery tube having a first tube end and a second tube end, the first tube end adapted for connection to the medicine bottle and the second tube end connected to the syringe needle assembly.

5. The automatically loading pole syringe of claim 5, further comprising an ink carriage for carrying an ink source, the ink carriage oriented to allow the ink source to discharge ink in a direction proximal to a needle carried by the syringe needle assembly.

6. The automatically loading pole syringe of claim 5, wherein the length of the syringe pole is adjustable.

7. The automatically loading pole syringe of claim 5, wherein the syringe needle assembly contains an interchangeable volume component.
